(19) United States
(12) Patent Application Publication
(10) Pub. No.: US 2019/0254273 A1
(43) Pub. Date: Aug. 22, 2019

(54) A CHEMICAL COMPOSITION TO STABILIZE EXTRACELLULAR VESICLES IN A BLOOD SAMPLE AND METHOD OF USE THEREOF

(71) Applicant: CFGEnome, LLC, Omaha, NE (US)

(72) Inventor: M. Rohan Fernando, Omaha, NE (US)

(73) Assignee: CFGEnome, LLC, Omaha, NE (US)

(21) Appl. No.: 16/306,799

(22) PCT Filed: Jun. 7, 2017
(86) PCT No.: PCT/US17/36413

§ 371 Date: Dec. 3, 2018

Related U.S. Application Data
(60) Provisional application No. 62/347,441, filed on Jun. 8, 2016.

(51) Int. Cl. A01N 1/02; G01N 1/40; G01N 33/49

(52) U.S. Cl. CPC A01N 1/0226; G01N 33/491; G01N 1/40

(57) ABSTRACT

Stabilizing compositions for stabilizing a post-phlebotomy, but pre-analysis, blood samples include a metabolic inhibitor, a protease inhibitor, a buffer system, an anticoagulant, and a solvent. The stabilizing compositions stabilize a post-phlebotomy blood sample to preserve the physiological state of the blood sample for later analysis. The analysis performed on the stabilized blood sample may determine the state of an analyte in a blood sample for diagnosis. The stabilizing compositions may stabilize the post-phlebotomy blood sample for at least 6 hours, and up to 672 hours. More preferably, the stabilizing composition has an undetectable level of formaldehyde before contact with the post-phlebotomy blood sample, so that cross linking of proteins and cross linking of proteins to nucleic acids in the post-phlebotomy blood sample is minimized. The stabilizing composition has a test sensitivity rate of 1 for up to 672 hours.

### REFERENCE TO RELATED APPLICATIONS

This application claims the benefit of U.S. Provisional Application No. 62/347,441, entitled “Stabilizing Composition and Methods of Use Thereof” filed Jun. 8, 2016, which is incorporated by reference in its entirety.

### BACKGROUND

Early detection of conditions of interest including diseases in humans is desired. For example, early detection of pregnancy, a chromosomal abnormality of a fetus, or cancer may lead to a better prognosis for the patient as intervention-type therapies may be used. However, the available early detection mechanisms that yield a diagnosis, and not merely a screening for the condition of interest, typically require an invasive procedure (e.g. amniocentesis, tumor biopsy, chronic villus sampling). Invasive procedures that provide diagnosis have an increased risk of adverse reactions such as infection, disease transmission, reaction to anesthesia administered during the procedure, and the like. Additionally, these invasive procedures are highly technical and are not adapted for widespread use. Due to the increased risks and complications with invasive procedures, it is desirable to have a noninvasive diagnostic test (e.g. diagnosis from a sample collected through a blood draw) to test for a condition of interest.

Diagnosis of a condition of interest is possible through a noninvasive diagnostic test performed on a blood sample by analysis of analytes, such as antibodies or biomarkers including proteins, lipids, DNA, mRNA and microRNA present in extracellular vesicles. Current clinical protocols for noninvasive detection of analytes in a blood sample requires immediate processing of the blood sample in order to obtain accurate and consistent diagnostic results. Diagnosis may be done by analysis of an analyte. Analysis of an analyte may be analysis of an analyte associated with the interior of an extracellular vesicle, analysis of an analyte associated with the exterior of an extracellular vesicle, or analysis of an analyte not associated with an extracellular vesicle. Delaying the processing of a blood sample allows the blood cells in the blood sample to release extracellular vesicles into the blood sample causing an artificial increase in non-target extracellular vesicles (e.g. background extracellular vesicles) that are not responsive to the condition of interest. The increase in background extracellular vesicles, artificially lowers the concentration (e.g. proportion) of the analyte in the post-phlebotomy blood sample.

FIG. 1 is an illustrative example of the increase in extracellular vesicles that occurs when processing of a blood sample is delayed for more than six hours versus immediate processing of a blood sample (e.g. processing within three hours of the blood draw). This increased release of background extracellular vesicles is believed triggered by exposing the cells in the blood sample to glucose deprivation and hypoxia, while the cells continue to metabolize. This “background” increase in extracellular vesicles may severely hamper the detection of the analyte of interest within a blood sample responsive to the condition of interest, and thus prevent the analysis from having sufficient accuracy and reproducibility for diagnosis.

Practical limitations such as distance from analysis laboratory, volume of samples for analysis, and the like, inhibit immediate processing of a blood sample. For example, many blood collection sites, in particular those in rural areas, are not equipped to process blood samples to analyze analytes for diagnosis. Instead blood samples are typically shipped from rural blood collection sites to a central analytical laboratory for analyte processing and analysis. Shipping samples from a sample collection site to a central laboratory is not practical or feasible given the current time constraints in current clinical analysis protocols. Further, in large clinical studies, the immediate processing of blood samples is not practical.

Current blood collection tubes for blood samples contain formaldehyde or chemicals that act as formaldehyde releasers. Formaldehyde causes significant damage to nucleic acids and proteins in the drawn blood, including those nucleic acids and proteins that are analytes, by forming protein to nucleic acid crosslinks and protein-to-protein crosslinks. As the time between collection and analysis of a sample increases, the damaging effect that formaldehyde has on the nucleic acids and proteins increases.

It is desirable to have compositions and methods that stabilize a post-phlebotomy (post-draw) blood sample for at least 6 hours at room temperature (from 18 to 25 degrees Celsius) so that the analyte of interest may be analyzed with sufficient accuracy and reproducibility for diagnosis of a condition of interest. It is further desirable that these compositions have undetectable levels of formaldehyde before combination with a post-phlebotomy blood sample and an undetectable level of formaldehyde up to 672 hours after contact with the post-phlebotomy blood sample because formaldehyde causes significant damage to nucleic acids and proteins through crosslinking.

### SUMMARY

Stabilizing compositions and methods of using the stabilizing composition are described.

In one aspect of the invention, a blood stabilizing composition, for stabilizing a post-phlebotomy blood sample prior to analysis includes a metabolic inhibitor, selected from the group consisting of diamide, azoester, 2-bromo-2-nitropropane-1, 3-diol, maleimide, N-ethylmaleimide, and combinations thereof, a protease inhibitor, selected from the group consisting of 6-aminohexanoic acid (amniocaprioic acid), 2-bromo-2-nitropropane-1, 3-diol, N-ethylmaleimide, ethylenediamine tetraacetic acid, Aprotinin, Benzamidine HCl, and combinations thereof, an acidic buffer capable of maintaining a pH from 3.0 to 6.5 or less, an anticoagulant, and a solvent.

In another aspect of the invention, a blood stabilizing composition, for stabilizing a post-phlebotomy blood sample prior to analysis includes from 3.3 to 6.6 grams per deciliter of a metabolic inhibitor, from 3.3 to 6.6 grams per deciliter of a protease inhibitor, from 1 to 2 grams per deciliter of a buffer, from 4.95 to 6.0 grams per deciliter of an anticoagulant, and a solvent.

In another aspect of the invention, a method for stabilizing a post-phlebotomy blood sample that includes contacting a post-phlebotomy blood sample with a stabilizing composition, wherein the stabilizing composition comprises a metabolic inhibitor, a protease inhibitor, a buffer, an anticoagulant, and a solvent, and storing the post-phlebotomy blood sample contacted with the stabilizing composition for at least 6 hours prior to analysis.

In another aspect of the invention, a method of analyzing a post phlebotomy blood sample to determine the presence or absence of a condition of interest, where the method includes stabilizing a post-phlebotomy blood sample, wherein the stabilizing comprises contacting the post-phlebotomy blood sample with a stabilizing composition, wherein, the stabilizing composition comprises a metabolic inhibitor, a protease inhibitor, a buffer, an anticoagulant, and a solvent storing the post-phlebotomy blood sample contacted with the stabilizing composition for at least 6 hours, separating a plasma having an analyte and a target extracellular vesicle from the post-phlebotomy blood sample contacted with the stabilizing composition, and analyzing the analyte to determine the presence or absence of the condition of interest.

In another aspect of the invention, a method of diagnosis of a disease including stabilizing a post-phlebotomy blood sample having an analyte, wherein the stabilizing comprises contacting the post-phlebotomy blood sample with a stabilizing composition, storing the post-phlebotomy blood sample having the analyte contacted with the stabilizing composition for at least 6 hours, analyzing the post-phlebotomy blood sample that has been stored for at least 6 hours where the analyte is maintained at a test sensitivity rate of 1.

The accompanying drawings, which are incorporated in and constitute a part of the specification, illustrate embodiments of the invention and together with the detailed description, serve to explain the principles of the invention.

### BRIEF DESCRIPTION OF THE DRAWINGS

The numerous advantages of the present invention may be better understood by those skilled in the art by reference to the accompanying figures in which:

FIG. 1 represents an illustrative example of the increase in extracellular vesicles that occurs when processing of a blood sample is delayed for more than six hours versus immediate processing of a blood sample (e.g. processing within three hours of the blood draw).

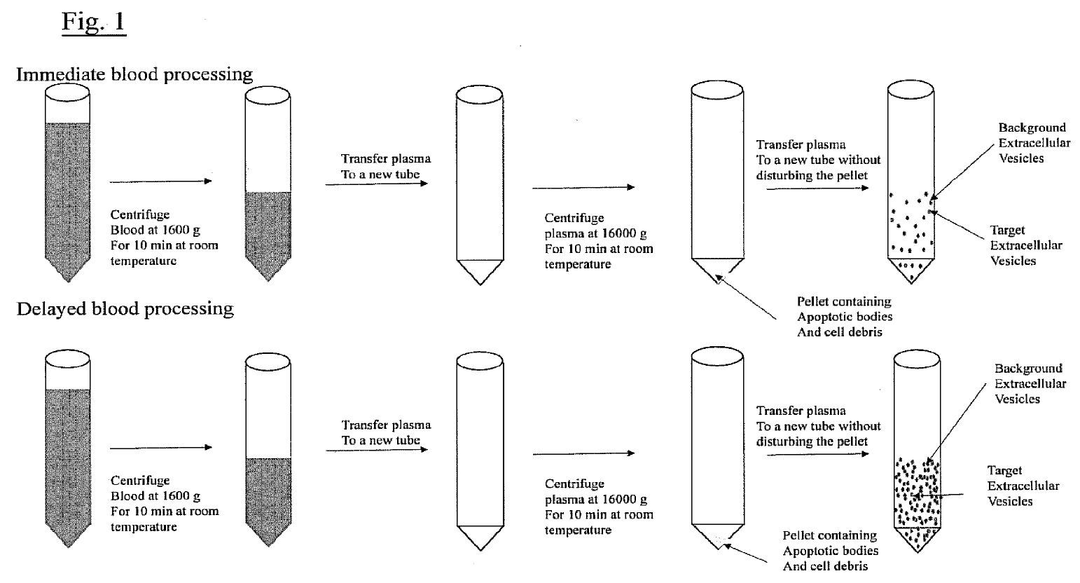

FIG. 2 represents a stabilizing composition tube 200.

FIG. 3 represents an undetectable level of formaldehyde in the stabilizing composition.

FIG. 4 represents a method 400 of stabilizing a post phlebotomy blood sample with a stabilizing composition tube.

FIG. 5 represents a method 500 of analyzing a post phlebotomy blood sample to determine the state of a condition of interest sufficient for diagnosis.

FIG. 6 represents a comparison of blood sample glucose concentrations determined from (1) a K3EDTA-only contacted post-phlebotomy blood sample and (2) a stabilizing composition contacted post-phlebotomy blood sample.

FIG. 7 represents a comparison of post-phlebotomy blood sample ras association domain-containing protein 1 (i.e. RASSF1A) DNA determined from (1) a post-phlebotomy blood sample contacted with the stabilizing composition and (2) a post-phlebotomy blood sample contacted with K3EDTA-only.

FIG. 8 represents a comparison of hemolysis between (1) a stabilizing composition contacted post-phlebotomy blood sample and (2) a post-phlebotomy blood sample contacted with K3EDTA-only.

FIG. 9 represents a comparison of plasma concentration of target extracellular vesicles and background extracellular vesicles (e.g. extracellular vesicles) between (1) a stabilizing composition contacted post-phlebotomy blood sample and (2) a K3EDTA-only contacted post-phlebotomy blood sample.

FIG. 10 represents a comparison of a protein concentration, a RNA concentration, and a DNA concentration determined from (1) a stabilizing composition contacted post-phlebotomy blood sample and (2) a K3EDTA-only contacted post-phlebotomy blood sample.

FIG. 11 represents a comparison of the test sensitivity of (1) a stabilizing composition contacted post-phlebotomy blood sample and (2) a K3EDTA-only contacted post-phlebotomy blood sample.

FIG. 12 represents the ability of the stabilizing composition to reduce protease activity in a post-phlebotomy blood sample.

### DETAILED DESCRIPTION

Stabilizing compositions for stabilizing a post-phlebotomy, but pre-analysis, blood samples include a metabolic inhibitor, a protease inhibitor, a buffer system, an anticoagulant, and a solvent. The stabilizing compositions stabilize a post-phlebotomy blood sample to preserve the physiological state of the blood sample for later analysis. The analysis performed on the stabilized blood sample may determine the state of an analyte in a blood sample for diagnosis.

The stabilizing compositions may stabilize the post-phlebotomy blood sample for at least 6 hours, and up to 672 hours. Preferably, the stabilizing composition has a level of formaldehyde of 0.005% (weight per volume) or less before contact with the post-phlebotomy blood sample. More preferably, the stabilizing composition has an undetectable level of formaldehyde before contact with the post-phlebotomy blood sample, so that cross linking of proteins and cross linking of proteins to nucleic acids in the post-phlebotomy blood sample is minimized.

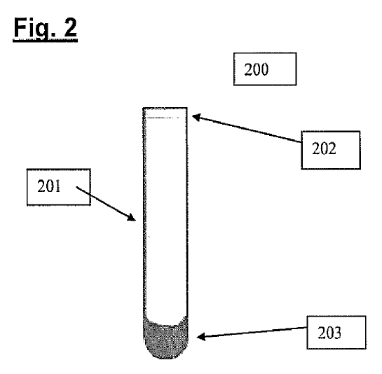

FIG. 2 represents a stabilizing composition tube 200. The stabilizing composition tube 200 includes a tube 201, a lid 202, and a stabilizing composition 203. The tube 201 of the stabilizing composition tube 200 may be a test tube of any size configured for holding liquids that is compatible with the stabilizing composition 203 and a post-phlebotomy blood sample. The tube 201 may be made of a non-reactive material, such as glass, plastic, metal, polypropylene, or ceramic.

The lid 202 of the stabilizing composition tube 200 is a lid configured for placement on the tube 201 to seal the tube 201 to keep liquid in the tube 201 when inverted and to keep contaminants that are airborne or the like out of the tube 201. The lid 202 may be of a non-reactive material including plastic, rubber, Teflon, metal, and combinations thereof. Most preferably, the lid 202 is configured to form a vacuum inside the tube 201 maintaining a sterile environment inside the tube 201.

The stabilizing composition 203 of the stabilizing composition tube 200 includes a metabolic inhibitor, a protease inhibitor, a buffer system, an anticoagulant, and a solvent. The stabilizing composition 203 stabilizes a post-phlebotomy blood sample for at least 6 hours. By stabilizing a post-phlebotomy blood sample, it is meant that after storage for at least 6 hours in the stabilizing composition tube 200 between 18 and 25 degrees Celsius, the analysis of an analyte in the post-phlebotomy blood sample to yield a diagnosis may be conducted.

The stabilizing composition has a level of formaldehyde of 0.005% (weight per volume) or less before contact with the post-phlebotomy blood sample. More preferably, the stabilizing composition has an undetectable level of formaldehyde before contact with the post-phlebotomy blood sample, so that cross linking of proteins and cross linking of proteins to nucleic acids in the post-phlebotomy blood sample is minimized.

The post-phlebotomy blood sample (not shown) may be collected from a human by venipuncture or other blood draw method. The post-phlebotomy blood sample may contain an analyte for analysis. The analyte may be an analyte associated with the interior of an extracellular vesicle, an analyte associated with the exterior of an extracellular vesicle, or an analyte not associated with an extracellular vesicle.

The metabolic inhibitor of the stabilizing composition 203 inhibits metabolism of cells in a post-phlebotomy blood sample. Inhibiting metabolism of cells in the post-phlebotomy blood sample limits the release of background extracellular vesicles from the cells in the post-phlebotomy blood sample. The metabolic inhibitor is chosen from the group consisting of diamide, azoester, 2-bromo-2-nitropropane-1, 3-diol (bronopol), maleimide, N-ethylmaleimide, and combinations thereof. The preferred metabolic inhibitors are bronopol and maleimide. The metabolic inhibitor constitutes from 3.3 to 6.6 g/dL of the stabilizing composition prior to contacting the stabilizing composition 203 with the post-phlebotomy blood sample. Without the metabolic inhibitor, the analysis would show an artificially high concentration (e.g. proportion) of the background extracellular vesicles.

The protease inhibitor of the stabilizing composition 203 inhibits proteolysis by protease enzymes in the post-phlebotomy blood sample. Inhibiting proteases in a post-phlebotomy blood sample reduces degradation of the analyte and thus stabilizes the analyte for analysis. The protease inhibitor is chosen from a group consisting of aminocaproic acid, bronopol, N-ethylmaleimide, ethylenediamine tetraacetic acid, Aprotinin, Benzamidine HCl, and combinations thereof. A preferred protease inhibitor is aminocaproic acid. The protease inhibitor constitutes from 3.3 to 6.6 g/dL of the stabilizing composition prior to contacting the stabilizing composition 203 with the post-phlebotomy blood sample. Without the protease inhibitor, the analysis would show an artificially low concentration of the analyte.

The buffer system of the stabilizing composition 203 reduces the hemolysis of cells in a post-phlebotomy blood sample. Reducing hemolysis of cells in a post-phlebotomy blood sample reduces degradation of the analyte in the post phlebotomy blood sample. The buffer system is acidic and capable of maintaining a pH from 3 to 6.5 and is chosen from the group consisting of tris-hydrochloride (tris-HCl), bis-tris-hydrochloride, N-(2-Acetamido)iminodiacetic acid, 2-(N-morpholino)ethanesulfonic acid, Tris(2-carboxyethyl) phosphine hydrochloride, dimethyl urea, imidazolidinyl urea, glycine, lysine, 2-mercaptoethanol and combinations thereof. The preferred buffer system may include prior to contacting the post-phlebotomy blood sample tris-HCl constituting from 1 g/dL to 2 g/dL of the stabilizing composition 203, dimethyl urea constituting from 3.3 g/dL to 13.2 g/dL of the stabilizing composition 203, 2-mercaptoethanol constituting from 0.132 g/dL to 0.264 g/dL of the stabilizing composition 203, and imidazolidinyl urea constituting from 0.165 g/dL to 3.3 g/dL of the stabilizing composition 203. Without the buffer system, the analysis would show an artificially low concentration of the analyte.

The anticoagulant of the stabilizing composition 203 reduces coagulation of the post-phlebotomy blood sample. Reducing coagulation of the post-phlebotomy blood sample stabilizes the analyte in the post-phlebotomy blood sample. The anticoagulant is selected from the group consisting of heparin, tri-potassium ethylenediamine tetraacetic acid (K₃EDTA), di-potassium ethylenediamine tetraacetic acid (K₂EDTA), citrate, oxalate, and combinations thereof. The preferred anticoagulants are K₃EDTA and K₂EDTA. The anticoagulant constitutes from 4.95 g/dL to 6.0 g/dL of the stabilizing composition 203 prior to contacting with the post-phlebotomy blood sample. Without the anticoagulant, the analysis would show an artificially low concentration of the analyte.

The solvent of the stabilizing composition 203 carries the metabolic inhibitor, the protease inhibitor, the anticoagulant, and the buffer system. The solvent is selected from the group consisting of sterilized distilled water, sterilized filtered water, and filtered and ozonated water. The preferred solvent is sterilized filtered water.

The stabilizing composition 203 preferably has a level of formaldehyde of 0.005% (weight/volume) or less prior to contacting the post-phlebotomy blood sample. Formaldehyde at 0.005% and less reduces damage to proteins and nucleic acids in a post-phlebotomy blood sample. Most preferably the stabilizing composition has an undetectable level of formaldehyde as measured by carbon 13 nuclear magnetic resonance spectroscopy (NMR) prior to contacting the post-phlebotomy blood sample.

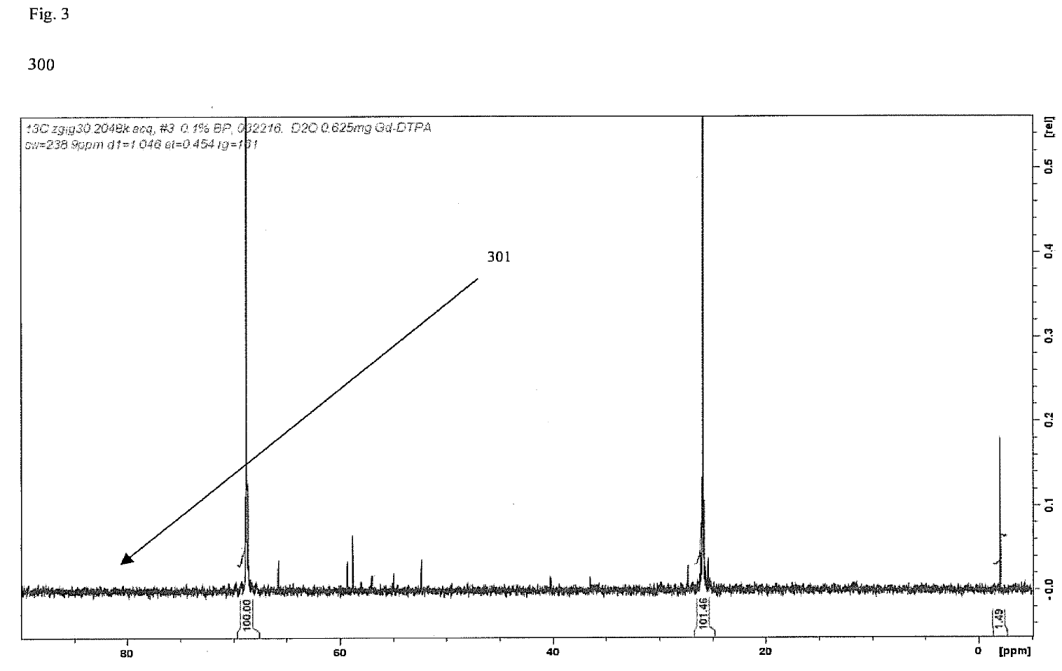

FIG. 3 illustrates an undetectable level of formaldehyde in the stabilizing composition. FIG. 3 shows a carbon 13 (C-13) NMR image 300 with deuterium oxide as the solvent of the stabilizing composition. The chemical shift (δ) 301 of hydrated formaldehyde (methylene glycol) in deuterium oxide should appear at 82.59 parts per million (ppm) relative to the Tetrahydrofuran-d₈ (THF) peak. The absence of the δ=82.59 ppm peak at 301 establishes that there is an undetectable level of hydrated formaldehyde in the stabilizing composition.

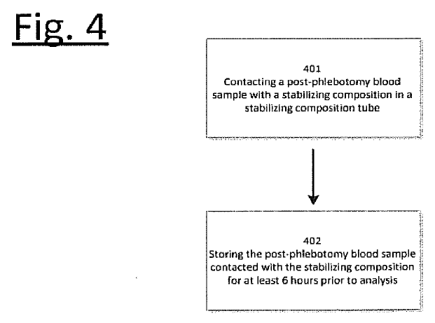

FIG. 4 illustrates a method 400 of stabilizing a post phlebotomy blood sample with a stabilizing composition tube. In 401, the post-phlebotomy blood sample is contacted with a stabilizing composition in the stabilizing composition tube. The contacting may include drawing the post-phlebotomy blood sample into the stabilizing composition tube. The contacting may further include inverting the blood collection tube one or more times.

In 402, the post-phlebotomy blood sample contacted with the stabilizing composition is stored for at least 6 hours prior to analysis. The post-phlebotomy blood sample may be stored for any period of time up to 672 hours. The post-phlebotomy blood sample may be stored at room temperature. Storing the post-phlebotomy blood sample, may include transporting the post-phlebotomy blood sample at room temperature.

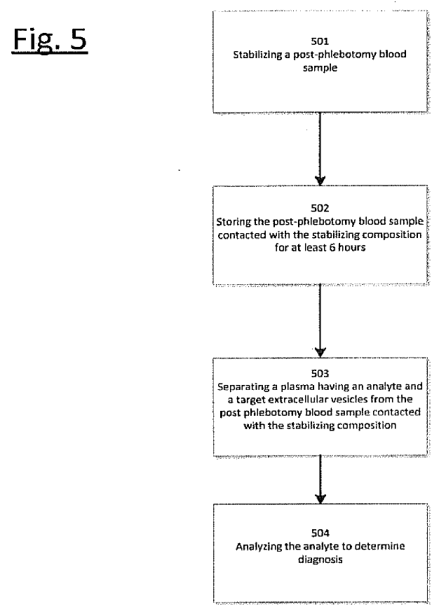

FIG. 5 illustrates a method 500 of analyzing a post phlebotomy blood sample to determine the state of a condition of interest sufficient for diagnosis. In 501, the post-phlebotomy blood sample is stabilized. The stabilizing may include contacting the post-phlebotomy blood sample with a stabilizing composition in a stabilizing composition tube. The contacting may include drawing the post-phlebotomy blood sample into the stabilizing composition tube. The contacting may further include inverting the stabilizing composition tube one or more times.

In 502, the post-phlebotomy blood sample contacted with the stabilizing composition is stored for at least 6 hours. The post-phlebotomy blood sample contacted with the stabilizing composition may be stored for any period of time up to 672 hours. The post-phlebotomy blood sample may be stored at room temperature. Storing the post-phlebotomy blood sample may include transporting the post-phlebotomy blood sample at room temperature.

In 503, the analyte is separated from the post-phlebotomy blood sample contacted with the stabilizing composition. The separating may include separating a plasma, where the plasma includes the analyte, target extracellular vesicles and background extracellular vesicles, from cells and cellular debris of the post-phlebotomy blood sample contacted with the stabilizing composition via two stage centrifugation. Two stage centrifugation may include centrifugation of the post-phlebotomy blood sample contacted with the stabilizing composition at 1600 (1.118×10⁻⁵) (e.g. ×g) for 10 minutes, transferring the plasma to a first centrifugation tube, centrifuging the transferred plasma at 16,000 g for 10 minutes to separate the plasma from a pellet containing cells and cellular debris, and transferring the plasma, which is substantially cell-free after two-stage centrifugation, without disturbing the pellet to a second centrifugation tube. The separated plasma includes the analyte, target extracellular vesicles, and background extracellular vesicles.

When the analyte is associated with the interior of an extracellular vesicle and when the analyte is associated with the exterior of an extracellular vesicle, 503 may further include, contacting the plasma having the analyte, target extracellular vesicles and background extracellular vesicles with an extracellular vesicle isolating reagent, such as sodium azide. The plasma having the analyte, target extracellular vesicles, and background extracellular vesicles is then incubated at 4 degrees Celsius for 30 minutes, followed by centrifugation at 10,000 g for 5 minutes at 4 degrees Celsius. After centrifugation the analyte, target extracellular vesicles, and background extracellular vesicles are in an extracellular vesicle pellet at the bottom of the second centrifugation tube. Plasma that is substantially free of the analyte, target extracellular vesicles and background extracellular vesicles is at the top of the second centrifugation tube.

When the analyte is associated with the interior of an extracellular vesicle and when the analyte is associated with the exterior of an extracellular vesicle, 503 may further include, contacting the extracellular vesicle pellet having the analyte, target and background extracellular vesicles with an analyte isolating reagent to isolate an analyte. When the analyte is DNA or RNA associated with the interior of a target extracellular vesicles, the contacting includes eluting the analyte with a silica-based membrane in a column. When the analyte is a protein associated with the interior of a target extracellular vesicles, the contacting includes solubilizing the protein associated with the interior of a target extracellular vesicle with a lysis buffer. When the analyte is a protein associated with the outside of a target extracellular vesicles, the extracellular vesicle pellet is contacted with antibodies specific to bind the protein.

In 504, the analyte is analyzed to determine a diagnosis associated with a condition of interest. For example, when the condition of interest is a genetic mutation, such as colon cancer, the determination will analyze the presence of the mutated gene in the post-phlebotomy blood sample. When chromosome aneuploidy is the condition of interest, for example trisomy 21, the determination will analyze the presence of an additional copy of chromosome 21 in the post-phlebotomy blood sample. When the condition of interest is the gender of an unborn baby, the determination will analyze the presence or absence of Y-chromosomal DNA in the post-phlebotomy blood sample. When the condition of interest is hyperglycemia or diabetes, the determination will analyze the glucose concentration of the post-phlebotomy blood sample.

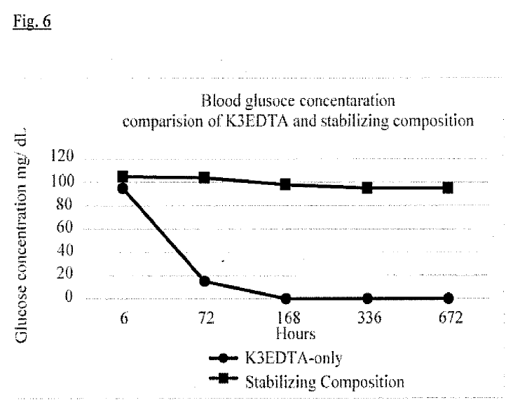

FIG. 6 compares post-phlebotomy blood sample glucose concentrations determined from (1) a post-phlebotomy blood sample contacted with K₃EDTA-only, and (2) a post-phlebotomy blood sample contacted with a previously described stabilizing composition. Glucose is an analyte not associated with a target extracellular vesical in a post-phlebotomy blood sample.

A stabilizing composition was chosen that prior to contact with the post-phlebotomy blood sample included 3.3 grams (g) per deciliter (dL) of bronopol, 3.3 g/dL of aminocaproic acid, a buffer system including 1 g/dL of Tris-HCl, 6.6 g/dL of dimethyl urea, 0.132 g/dL of 2-mercaptoethanol, 1.65 g/dL of imidazolidinyl urea, 5.61 g/dL of K₃EDTA, and 3,301.6 g/dL of sterile distilled water.

After combining this stabilizing composition with the post-phlebotomy blood sample, the mixture included 0.1 grams (g) per deciliter (dL) of bronopol, 0.1 g/dL of 6-aminohexanoic acid (aminocaproic acid), the buffer system of 0.03 g/dL of Tris-HCl buffer, 0.2 g/dL of dimethyl urea, 0.004 g/dL of 2-mercaptoethanol, and 0.05 g/dL of imidazolidinyl urea, 0.17 g/dL of K₃EDTA, and 99.146 g/dL of sterile distilled water. While a specific stabilizing composition was used in this instance, other stabilizing compositions also could be used.

A post-phlebotomy blood sample contacted with K₃EDTA was prepared by contacting the post-phlebotomy blood sample in a collection tube constituting from 1.2 to 2 mg of K₃EDTA in spray-dried form (e.g. K₃EDTA-only tube). A post-phlebotomy blood sample contacted with a K₃EDTA-only tube is a K₃EDTA-only contacted post-phlebotomy blood sample.

Glucose concentrations were determined for each post-phlebotomy blood sample over a period of 672 hours using a glucometer to measure grams of glucose per deciliter in the substantially cell-free plasma layer isolated from each sample.

The glucose concentration of the K₃EDTA-only contacted post-phlebotomy blood sample showed a 84% decrease in glucose concentration from hour 6 to hour 72. From hour 6 to hour 168 the K₃EDTA-only contacted post-phlebotomy blood sample showed a 100% decrease in glucose concentration. From hour 168 to hour 672, the glucose remained constant at a 0% concentration. This suggests that the cells of the K₃EDTA-only contacted post-phlebotomy blood sample continued cellular metabolism until substantially all of the glucose in the K₃EDTA-only contacted post-phlebotomy blood sample was consumed by the cells.

The glucose concentration of the stabilizing composition contacted post-phlebotomy blood sample showed a 5.7% decrease from hour 6 to hour 72, a 15.6% decrease from hour 72 to hour 168, a 8.4% decrease from hour 168 to hour 336, and a 7.7% decrease from hour 336 to hour 672. Taken together, an approximate 9.35% decrease from hour 6 to hour 672 was observed. Thus, metabolism was inhibited approximately 93% by the stabilizing composition in comparison to the K₃EDTA-only contacted sample. The stabilizing composition contacted blood sample could provide an accurate glucose determination up to at least 672 hours after the blood was drawn.

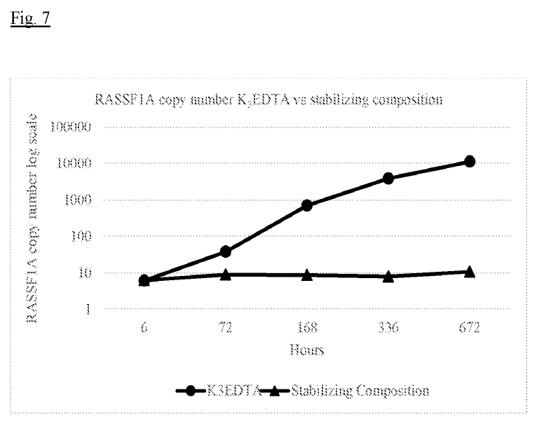

FIG. 7 compares post-phlebotomy blood sample ras association domain-containing protein 1 (i.e. RASSF1A) DNA determined from (1) a post-phlebotomy blood sample contacted with the previously described stabilizing composition and (2) a post-phlebotomy blood sample contacted with K₃EDTA-only. Hypo-methylated RASSF1A is associated in background extracellular vesicles. Hyper-methylated RASSF1A is an analyte associated with the interior of a target extracellular vesicle. Cells in a post-phlebotomy blood sample release background extracellular vesicles, thus this example demonstrates the ability of the described stabilizing compositions to suppress release of background extracellular vesicles in relation to a K₃EDTA-only contacted sample. In this instance the stabilizing composition of FIG. 6 was used to stabilize the post-phlebotomy blood sample, but other stabilizing composition may be used.

The stabilizing composition contacted post-phlebotomy blood sample showed a reduction in RASSF1A DNA over the K₃EDTA-only contacted post-phlebotomy blood sample of 380% after 72 hours, 4,525% over 168 hours, 17,430% over 336 hours, and 36,4315% over 672 hours. Thus, the stabilizing composition contacted post-phlebotomy blood sample significantly reduces an artificially low determined level of hyper-methylated RASSF1A from a post-phlebotomy blood sample in relation to the K₃EDTA-only contacted post-phlebotomy blood sample. Unlike the K₃EDTA-only contacted post-phlebotomy blood sample, the stabilizing composition contacted post-phlebotomy blood sample may provide an accurate determination of hyper-methylated RASSF1A.

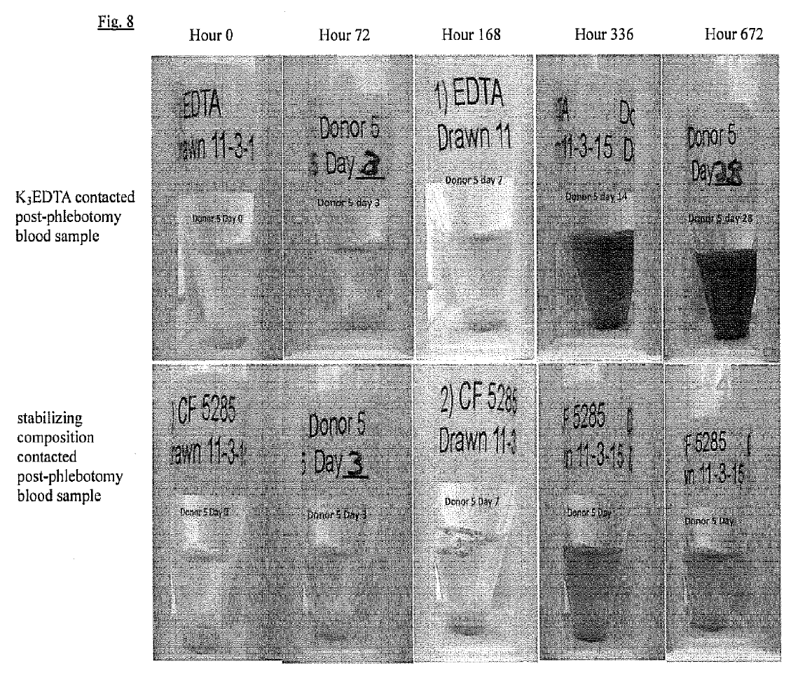

FIG. 8 compares hemolysis between (1) a stabilizing composition contacted post-phlebotomy blood sample and (2) a post-phlebotomy blood sample contacted with K₃EDTA-only. In this instance the stabilizing composition of FIG. 6 was used to stabilize the post-phlebotomy blood sample, but other stabilizing compositions may be used.

The K₃EDTA-only contacted post-phlebotomy blood sample showed indication of hemolysis at hour 168 with such indications increasing through hour 672. The stabilizing composition contacted post-phlebotomy blood sample showed slight indications of hemolysis after 336 hours and 672 hours. Comparing the stabilizing composition contacted post-phlebotomy blood sample and the K₃EDTA-only contacted post-phlebotomy blood sample, the K₃EDTA-only contacted post-phlebotomy blood sample displayed significant hemolysis at 336 hours, as compared to the stabilizing composition contacted post-phlebotomy blood sample which displayed slight hemolysis at 336 hours. Thus, the stabilizing composition inhibits hemolysis in a post-phlebotomy blood sample.

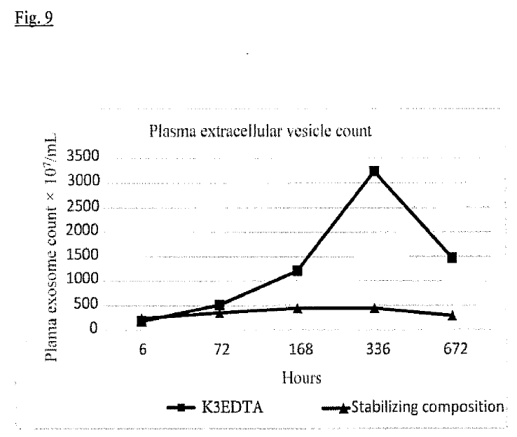

FIG. 9 compares plasma concentration of target extracellular vesicles and background extracellular vesicles (e.g. extracellular vesicles) between a stabilizing composition contacted post-phlebotomy blood sample and a K₃EDTA-only contacted post-phlebotomy blood sample. In this instance the stabilizing composition of FIG. 6 was used to stabilize the post-phlebotomy blood sample, but other stabilizing compositions may be used.

The K₃EDTA-only contacted post-phlebotomy blood sample showed an increase of 45% of extracellular vesicles at hour 72 as compared to the stabilizing composition contacted post-phlebotomy blood sample. The K₃EDTA-only contacted post-phlebotomy blood sample showed an increase of 173% of extracellular vesicles at hour 168 as compared to the stabilizing composition contacted post-phlebotomy blood sample. The K₃EDTA-only contacted post-phlebotomy blood sample showed an increase of 627% of extracellular vesicles at hour 336 as compared to the stabilizing composition contacted post-phlebotomy blood sample. The K₃EDTA-only contacted post-phlebotomy blood sample showed an increase of 404% of extracellular vesicles at hour 672 as compared to the stabilizing composition post-phlebotomy blood sample. Thus, the stabilizing composition reduced the amount of background extracellular vesicles in a post-phlebotomy blood sample in relation to the K₃EDTA-only contacted post-phlebotomy blood sample. The stabilizing composition contacted blood sample may provide an accurate determination of an analyte associated with the interior of a target extracellular vesicle or an analyte associated with the exterior of a target extracellular vesicle.

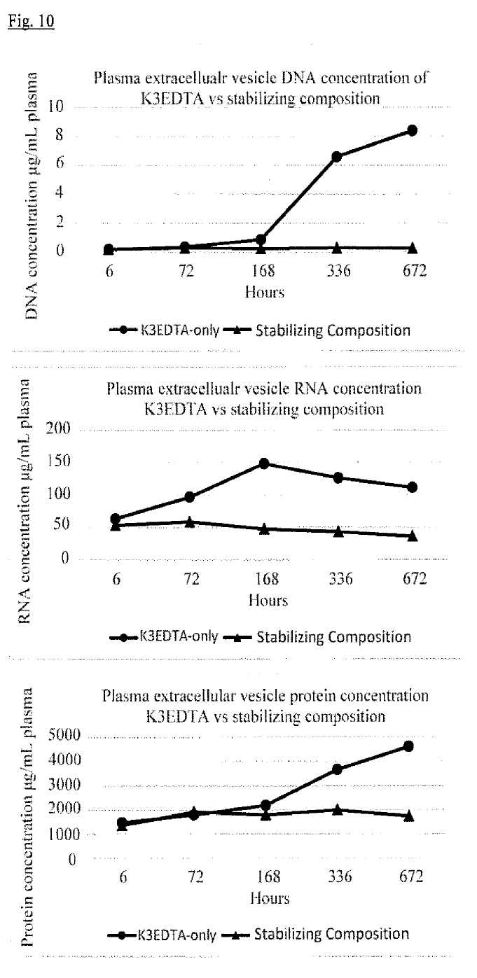

FIG. 10 compares a protein concentration, a RNA concentration, and a DNA concentration determined from a stabilizing composition contacted post-phlebotomy blood sample and a K₃EDTA-only contacted post-phlebotomy blood sample. In this instance the stabilizing composition of FIG. 6 was used to contact the post-phlebotomy blood sample, but other stabilizing compositions may be used.

The percentages used with respect to FIG. 10 are expressed in the context of the K₃EDTA-only contacted blood sample being the baseline measurement, where the stabilizing composition yields a concentration of protein, RNA, or DNA that is less than the baseline measurement. The stabilizing composition contacted post-phlebotomy blood sample showed a reduction of the protein concentration of 22% after 72 hours, 48% after 168 hours, 149% after 336 hours, and 213% after 672 hours as compared to the K₃EDTA-only contacted second post-phlebotomy blood sample. Comparatively, the stabilizing composition contacted post-phlebotomy blood sample showed a reduction in the RNA concentration of 54% after 72 hours, 135% after 168 hours, 100% after 336 hours, and 76% after 672 hours, as compared to the K₃EDTA-only contacted post-phlebotomy blood sample. Comparatively, the stabilizing composition contacted post-phlebotomy blood sample showed a reduction in the DNA concentration 118% after 72 hours, 438% after 168 hours, 4164% after 336 hours, and 5339% after 672 hours as compared to the K₃EDTA-only post-phlebotomy blood sample. Thus, the stabilizing composition inhibits the release of background extracellular vesicles in a post-phlebotomy blood sample and reduces cell degradation in a post-phlebotomy blood sample.

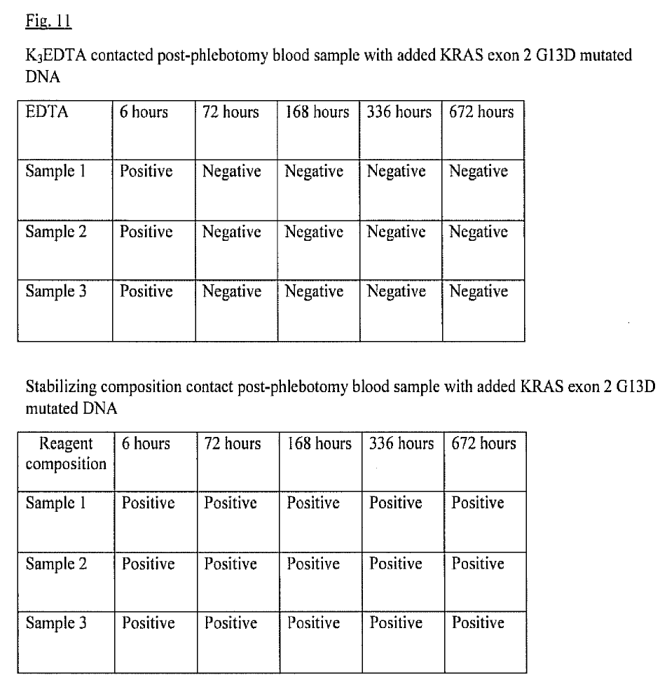

FIG. 11 compares the test sensitivity of a stabilizing composition contacted post-phlebotomy blood sample and a K₃EDTA-only contacted post-phlebotomy blood sample. The post-phlebotomy blood samples had the same volume of extracellular vesicles from the human colon cancer cell line HCT 116 that carry kirston rat sarcoma viral oncogene homolog (KRAS) exon 2 G13D with a heterozygous mutation (e.g. KRAS mutation) at a concentration of 200 copies/mL added. In this instance the stabilizing composition of FIG. 6 was used to contact the post-phlebotomy blood sample, but other stabilizing compositions may be used.

The stabilizing composition contacted post-phlebotomy blood samples with added KRAS mutation extracellular vesicles were tested for sensitivity to the KRAS mutation using real time polymerase chain reaction (PCR) detection. The stabilizing composition contacted post-phlebotomy blood samples with added KRAS mutation extracellular vesicles showed a test sensitivity of 1 after 6, 72, 168, 336, and 672 hours. The K₃EDTA-only contacted post-phlebotomy blood sample with added KRAS mutation extracellular vesicles were tested for sensitivity to the KRAS mutation using real time PCR detection. The K₃EDTA-only contacted post-phlebotomy blood sample with added KRAS mutation extracellular vesicles showed a test sensitivity of 1 only after 6 hours. The stabilizing composition extends test sensitivity of 1 up to 672 hours as compared to the K₃EDTA-only that only provides a test sensitivity of 1 up to 6 hours.

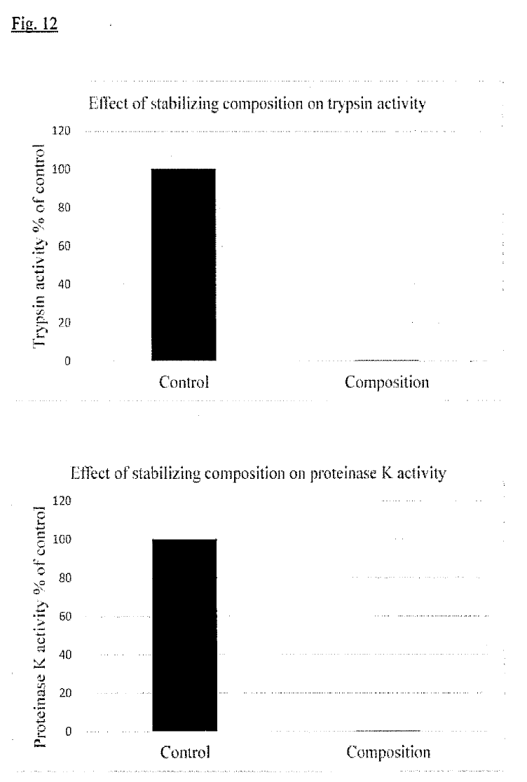

FIG. 12 demonstrates the ability of the stabilizing composition to reduce protease activity in a post-phlebotomy blood sample. The effect of the stabilizing composition on protease activity was measured by determining enzyme activity of the proteases trypsin and proteinase K in a stabilizing composition contacted purified trypsin sample, a stabilizing composition contacted purified proteinase K sample, a phosphate buffered saline (PBS) contacted purified trypsin sample, and a PBS contacted purified proteinase K composition sample. In this instance the stabilizing composition of FIG. 6 was used, but other stabilizing compositions may be used. Further, the PBS contacted trypsin and proteinase K samples are labeled as “control” in FIG. 12

The stabilizing composition contacted purified trypsin sample shows a 95% reduction in trypsin activity. The stabilizing composition contacted purified proteinase K sample shows a 95% reduction in proteinase K activity after contact and incubation with the stabilizing composition. The PBS contacted purified trypsin and PBS contacted purified proteinase K sample each show no statistically significant reduction in either trypsin or proteinase K activities. This demonstrates that the trypsin and proteinase K activity is substantially inhibited in a stabilizing composition contacted post-phlebotomy blood sample. Substantial inhibition of protease activity limits degradation of analytes.

The following definitions are provided to assist in a consistent understanding of the specification and claims.

An analyte may be an antibody, protein, lipid, DNA, mRNA microRNA, cfDNA, membrane receptors, adhesion molecules, cytokines, chemokines, and growth factors. An analyte may be associated with the interior of a target extracellular vesicle. An analyte may be associated with the exterior of a target extracellular vesicle, or an analyte may not be associated with an extracellular vesicle.

Target extracellular vesicles are extracellular vesicles (e.g. microvesicles and exosomes) produced by a cell associated with a condition of interest. The target extracellular vesicles are produced by cells that indicate the condition of interest. The target extracellular vesicles may be associated with analytes, which reflect the cellular origin and the physiological state of the cell from which the analyte originates.

Background extracellular vesicles in a sample are extracellular vesicles produced by cells not associated with the condition of interest (e.g. maternal cells, or normal, healthy human cells) and include the background extracellular vesicles present in the sample at the time the sample is drawn and the background extracellular vesicles produced between the time the sample is drawn and analysis of the sample.

Diagnosis is analysis of a post-phlebotomy blood sample that produces a determination of the state of a condition of interest where the post-phlebotomy blood sample has been stored for at least 6 hours and the test sensitivity for the analyte is maintained at a rate of 1, where test sensitivity is the rate of true positive results divided by the sum of true positive results plus false negative results. The state of a condition of interest may be the presence or absence of the condition of interest.

1. A blood stabilizing composition, for stabilizing a post-phlebotomy blood sample prior to analysis, comprising:
a metabolic inhibitor, selected from the group consisting of diamide, azoester, 2-bromo-2-nitropropane-1, 3-diol, maleimide, N-ethylmaleimide, and combinations thereof;
a protease inhibitor, selected from the group consisting of 6-aminohexanoic acid, 2-bromo-2-nitropropane-1, 3-diol, N-ethylmaleimide, ethylenediamine tetraacetic acid, Aprotinin, Benzamidine HCl, and combinations thereof;
an acidic buffer capable of maintaining a pH from 3.0 to 6.5 or less;
an anticoagulant; and
a solvent.

2. The stabilizing composition of claim 1, wherein
the metabolic inhibitor is selected from the group consisting of diamide, azoester, 2-bromo-2-nitropropane-1, 3-diol, maleimide, N-ethylmaleimide, and combinations thereof;
the protease inhibitor is selected from the group consisting of 6-aminohexanoic acid, 2-bromo-2-nitropropane-1, 3-diol, N-ethylmaleimide, ethylenediamine tetraacetic acid, Aprotinin, Benzamidine HCl, and combinations thereof;
the buffer is selected from the group consisting of tris-hydrochloride, bis-tris-hydrochloride, N-(2-Acetamido)iminodiacetic acid, 2-(N-morpholino)ethanesulfonic acid, Tris(2-carboxyethyl) phosphine hydrochloride, dimethyl urea, imidazolidinyl urea, glycine, lysine, 2-mercaptoethanol and combinations thereof;
the anticoagulant is selected from the group consisting of heparin, tri-potassium ethylenediamine tetraacetic acid, di-potassium ethylenediamine tetraacetic acid citrate, oxalate, and combinations thereof; and
the solvent is selected from the group consisting of sterilized distilled water, sterilized filtered water, and filtered ozonized water.

3. The stabilizing composition of claim 1, wherein
the metabolic inhibitor is 2-bromo-2-nitropropane-1, 3-diol;
the protease inhibitor is 6-aminohexanoic acid;
the acidic buffer is tris-hydrochloride, dimethyl urea, 2-mercaptoethanol, and imidazolidinyl urea;
the anticoagulant is ethylenediamine tetraacetic acid; and
the solvent is sterilized filtered water.

4. A blood stabilizing composition, for stabilizing a post-phlebotomy blood sample prior to analysis, comprising:
from 3.3 to 6.6 grams per deciliter of a metabolic inhibitor;
from 3.3 to 6.6 grams per deciliter of a protease inhibitor;
from 1 to 2 grams per deciliter of an acidic buffer;
from 4.95 to 6.0 grams per deciliter of an anticoagulant; and
a solvent.

5. The stabilizing composition of claim 4, wherein the stabilizing composition has a pH from 3 to 6.5.

6. The stabilizing composition of claim 4, wherein the stabilizing composition has an undetectable amount of formaldehyde when analyzed by carbon 13 nuclear magnetic resonance imaging in deuterium oxide from 18 to 25 degrees Celsius.

7. A method for stabilizing a post-phlebotomy blood sample, comprising:
contacting a post-phlebotomy blood sample with a stabilizing composition,
wherein the stabilizing composition comprises a metabolic inhibitor, a protease inhibitor, a buffer, an anticoagulant, and a solvent;
storing the post-phlebotomy blood sample contacted with the stabilizing composition for at least 6 hours prior to analysis.

8. The method of claim 7, where the storing further comprises storing the post-phlebotomy blood sample contacted with the stabilizing composition at a temperature from 10 to 30 degrees Celsius.

9. The method of claim 8, where the storing is for at least 72 hours.

10. The method of claim 8, where the storing is for at least 168 hours.

11. The method of claim 8, where the storing is for at least 336 hours.

12. The method of claim 8, where the storing is for at least 672 hours.

13. A method of analyzing a post phlebotomy blood sample to determine the presence or absence of a condition of interest, the method comprising:
stabilizing a post-phlebotomy blood sample,
wherein the stabilizing comprises contacting the post-phlebotomy blood sample with a stabilizing composition,
wherein, the stabilizing composition comprises a metabolic inhibitor, a protease inhibitor, a buffer, an anticoagulant, and a solvent
storing the post-phlebotomy blood sample contacted with the stabilizing composition for at least 6 hours;
separating a plasma having an analyte and a target extracellular vesicle from the post-phlebotomy blood sample contacted with the stabilizing composition; and
analyzing the analyte to determine the presence or absence of the condition of interest.

14. The method of claim 13, where the separating comprises separating the plasma including the analyte and the target extracellular vesicles from cells and cellular debris of the post-phlebotomy blood sample contacted with the stabilizing composition, wherein the separating includes two stage centrifugation.

15. The method of claim 13, where the separating further comprises contacting the plasma having the analyte and the target extracellular vesicle with an extracellular vesicle isolating reagent, when the analyte is an analyte associated with the interior of a target extracellular vesicle and an analyte associated with the exterior of a target extracellular vesicle.

16. The method of claim 15, where the separating further comprises eluting with a silica-based membrane for binding the analyte, when the analyte is associated with the interior of the target extracellular vesicle.

17. The method of claim 15, where the separating comprises solubilizing the analyte with a lysis buffer, when the analyte is associated with the interior of the target extracellular vesicle.

18. The method of claim 15, where the separating further comprises binding an antibody specific to the target analyte, when the target analyte is associated with the exterior of the target extracellular vesicle.

19. A method of diagnosis of a disease comprising
stabilizing a post-phlebotomy blood sample having an analyte,
wherein the stabilizing comprises contacting the post-phlebotomy blood sample with a stabilizing composition,
storing the post-phlebotomy blood sample having the analyte contacted with the stabilizing composition for at least 6 hours,
analyzing the post-phlebotomy blood sample that has been stored for at least 6 hours where the analyte is maintained at a test sensitivity rate of 1.
